(19) United States
(12) Patent Application Publication
(10) Pub. No.: US 2011/0060202 A1
(43) Pub. Date: Mar. 10, 2011

(54) DEHYDRATION DETECTOR USING MICRO-NEEDLES

(76) Inventor: Seth Adrian Miller, Englewood, CO (US)

(21) Appl. No.: 12/555,543

(22) Filed: Sep. 8, 2009

(51) Int. Cl. A61B 5/145; G01R 27/08

(52) U.S. Cl. 600/345; 324/694

(57) ABSTRACT

Examples are generally described that include an array of micro-needles. A capillary may be coupled to the array of micro-needles and configured to receive fluid from at least a portion of the array. A conductivity sensor may be arranged to measure conductivity of the fluid within the capillary, and a pump may be arranged to move fluid through the capillary. A processor may arranged to receive signals indicative of conductivity from the conductivity sensor and identify a dehydration condition based at least in part on a change in the signals indicative of conductivity.

### BACKGROUND

Dehydration may be a concern for participants in competitive athletics, workers engaged in outdoor physical activity, soldiers participating in military operations, or any of a variety of other situations. Acute dehydration can be life-threatening, but significant reductions in performance due to less severe dehydration may also occur. Existing methods to detect dehydration, such as urine analysis, may be time-consuming and interfere with a subject's activities.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are, therefore, not to be considered limiting of its scope, the disclosure will be described with additional specificity and detail through use of the accompanying drawings, in which:

FIG. 1 is a schematic illustration of an example of a system for detecting dehydration in accordance with at least some embodiments of the present disclosure.

FIG. 2 is a flowchart of an example of monitoring a subject for dehydration in accordance with at least some embodiments of the present disclosure.

FIG. 3 is a block diagram illustrating an example computing device 900 that is arranged for monitoring for dehydration in accordance with at least some embodiments of the present disclosure.

FIG. 4 is a block diagram illustrating an example computer program product 600 that is arranged to store instructions for monitoring a fluid for dehydration in accordance with at least some embodiments of the present disclosure.

### DETAILED DESCRIPTION

The following detailed description, reference is made to the accompanying drawings, which form a part hereof. In the drawings, similar symbols typically identify similar components, unless context dictates otherwise. The illustrative examples described in the detailed description, drawings, and claims are not meant to be limiting. Other examples may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are implicitly contemplated herein.

This disclosure is drawn, inter alia, to methods, systems, devices, and/or apparatus generally related to detection of dehydration using micro-needles. Some example devices may include a capillary, an array of micro-needles, a conductivity sensor, a pump, and a processor. The capillary may be coupled to the array of micro-needles and configured to receive fluid from at least a portion of the array. The conductivity sensor may be arranged to measure conductivity of the fluid within the capillary, and the pump may be arranged to move fluid through the capillary. The processor may arranged to receive signals indicative of conductivity from the conductivity sensor and identify a dehydration condition based at least in part on a change in the signals indicative of conductivity.

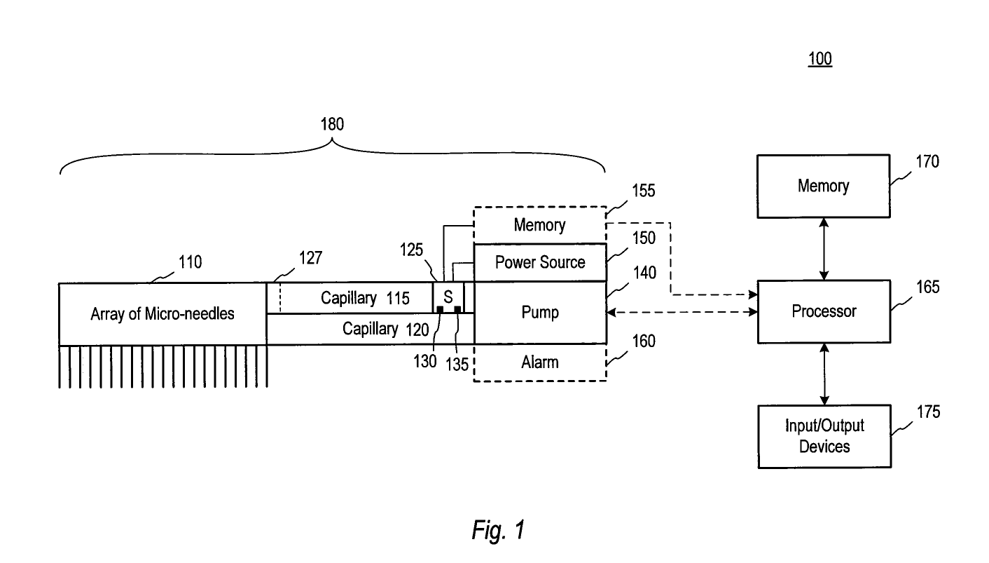

FIG. 1 is a schematic illustration of an example of a system for detecting dehydration in accordance with at least some embodiments of the present disclosure. The system 100 includes an array of micro-needles 110 coupled to capillaries 115 and 120. A conductivity sensor 125 may be positioned in the capillary 115 and may include two electrodes 130 and 135. The capillaries 115 and 120 may also include optional filters, such as the filter 127. A pump 140 may be coupled to the capillaries 115 and 120 and may be used to move fluid through one or both of the capillaries 115 and 120. A power source 150 may be adapted to provide power to the conductivity sensor 125 and the pump 140, and may also be arranged to provide power to other components. A memory 155 may be coupled to the conductivity sensor 125 and may be configured to store conductivity readings taken by the conductivity sensor 125. An alarm 160 may also be provided to alert a user to a detected dehydration condition. A processor 165 may be coupled to the memory 155, conductivity sensor 125, or both, and arranged to receive conductivity measurements and provide an indication of dehydration based at least in part on a change in the conductivity of the fluid. A memory 170 may be adapted to store computer executable instructions for the processor 165. Input/output devices 175 may be arranged to interface to the processor 165.

Utilizing the apparatus 100, a fluid, such as interstitial fluid, blood, plasma, sweat, or combinations of those fluids, may be drawn through the micro-needle array 110 from a subject, such as but not limited to a human or other live body. The conductivity of the fluid drawn from the needle array may be measured by the conductivity sensor 125. The pump 140 may be adapted to continually pull fluid through the capillary 115 so that new measurements can be obtained.

The array of micro-needles 110 may include any number of needles in any physical configuration, and the term ‘array’ is not intended to be limited to a specific pattern. In a simple example, a grid pattern may be used. In some examples, the array of micro-needles 110 may be arranged in either a one dimensional (1-D) or two-dimensional pattern, which may be either linear, non-linear or some other combination thereof. In still other examples, the array of micro-needles 110 may be arranged in a random or pseudo random configuration.

Fluid may enter the needles by an internal pressure of the subject, such as a human, or through the force of the pump 140, or both. Micro-needles may be formed utilizing microfabrication or micro-electro mechanical system fabrication technologies, or both, including but not limited to, material growth or deposition, photolithography, and wet and dry etching. Micro-needles may have a microscale diameter, such as a diameter between about 1 μm and about 1,000 μm, in some examples between about 1 μm and about 10 μm, in some examples between about 10 μm and about 100 μm, in some examples between about 100 μm and about 500 μm, in some examples between about 500 μm and about 1,000 μm, in some examples between about 20 μm and about 800 μm, in some examples between about 50 μm and about 500 μm, and in some examples between about 100 μm and about 300 μm.

The array of micro-needles 110 may be applied as a patch to the skin of a subject, for example to sample blood, plasma, sweat, interstitial fluid, or combinations of those fluids. Suitable micro-needles have been developed, typically for use in drug delivery, and examples may be found at http://www.eurekalert.org/pub releases/2008-02/uok-med020408.php, and J. G. E. Han et al., “Silicon Micromachined Hollow Microneedles for Transdermal Liquid Transport,” Journal of Micromechanical Systems, vol. 12(6) (2003), both of which are hereby incorporated by reference in their entirety for any purpose.

The capillaries 115 and 120 may be implemented as respective channels micromachined into a substrate, such as, but not limited to, a plastic or glass substrate. The capillaries 115 and 120 may be configured to draw liquid into their entry points. In this manner, the substrate defining the channels used as the capillaries 115 and 120 may be coated with an appropriate coating configured to wick in fluids, in some examples. The capillaries 115 and 120 may also be fabricated using microfabrication or micro-electro mechanical system fabrication technologies, or both, including but not limited to, material growth or deposition, photolithography, and wet and dry etching. Capillaries as described herein may typically then have a microscale width, such as a width between about 1 μm and about 1,000 μm, in some examples between about 1 μm and about 10 μm, in some examples between about 10 μm and about 100 μm, in some examples between about 100 μm and about 500 μm, in some examples between about 500 μm and about 1,000 μm, in some examples between about 20 μm and about 800 μm, in some examples between about 50 μm and about 500 μm, and in some examples between about 100 μm and about 300 μm. In some examples, a width of one or more capillaries may be selected based on properties of the fluid to be drawn into the capillary, and may be selected to assist in the drawing of fluid into the capillary through capillary action, although capillary action is not required. Two capillaries 115 and 120 are shown in FIG. 1, although any number may be used, including one capillary. In some examples, multiple capillaries may be advantageous to improve the efficiency of the pump described below. In some examples, one or more of the capillaries may be implemented as, or coupled to, a chamber or reservoir into which the fluid from the micro-needles is drawn.

The filter 127 may be provided at an inlet of one or more of the capillaries. The filter 127 may be implemented as a thin plug of fabric that is adapted to filter and wick fluid, such as plasma, to the capillary. The fabric may be selected so as to not alter the ionic concentration or pH of the fluid. Alternatively, more sophisticated methods of filtration such as diffusive filters can be used. Suitable diffusive filters are described for example at N. Pamme, “Continuous flow separations in microfluidic devices,” Lab Chip, 7, pp. 1644-1659 (2007), which is incorporated by reference herein in its entirety for any purpose.

The pump 140 may be implemented as an electro-osmotic pump and may be configured to pump continuously. The continuous pumping may allow fluid to be sampled from a subject in real time, and analysis may also occur in real-time. The electro-osmotic pump may be placed in one or more of the capillaries 115 and 120, and may be implemented utilizing the pair of electrodes 130 and 135 that may also be utilized for the conductivity sensor 125. The electrodes may be positioned in direct contact with the fluid in the capillary, although direct contact may not be required and may be undesirable if the electrode material is not compatible with the fluid. Other types of pumps may be used in other examples.

The conductivity sensor 125 may also be implemented as a pair of electrodes configured to measure a conductivity of a fluid in the capillary 115. In one example, the electrodes 130 and 135 used to form the conductivity sensor 125 may also form all or part of the pump 140. By applying an AC signal to the electrodes 130 and 135, for example an AC signal with an amplitude of 100 mV and a frequency of 10 kHz, the real and imaginary impedance of a fluid in the capillaries may be measured. Other voltage amplitudes and frequencies may also be used. Dehydration may result in a change in concentration of electrolytes in the fluid. Accordingly, a detected change in conductivity may be used to generate a signal indicative of dehydration.

The processor 165 may accordingly be configured to monitor a signal indicative of fluid conductivity generated by the conductivity sensor 125. The processor 165 may be external to the apparatus 180 including the micro-needle array 110, or may be integrated in some examples. In some examples, the processor 165 may be a low-power digital signal processing (DSP) unit. The processor 165 may be configured to generate a signal indicative of dehydration that is, at least in part, responsive to a change in conductivity of the fluid as represented by a change in the signal indicative of conductivity generated by the conductivity sensor 125. The processor 165 may be programmed by instructions encoded on the memory 170. Alternatively or in addition, the memory 155 may be provided in the apparatus 180 locally and configured to store one or more signals indicative of conductivity generated by the sensor 125 and may later communicate those signals through a transmitter or other communication link to the processor 165. The input/output devices 175 may also be provided as an interface to the processor 165, memory, or both, as will be described further below. In some examples, the processor 165 may be configured to determine that the fluid in the capillary has decreased in resistance, and generate a signal indicative of dehydration. In some additional examples, the signal indicative of dehydration may not be generated unless the change in resistance is greater than a predetermined level, which in some examples may be considered a noise threshold level.

The alarm 160 may be arranged to alert the subject, or other user of the apparatus 180, to the dehydration condition. The alarm 160 may be implemented as an audible alarm, such as, but not limited to, a clicking or buzzing noise, a visual alarm, such as, but not limited to, a flashing LED, or other perceptible mechanism to communicate the presence of a dehydration condition. An indication of the dehydration condition may also be displayed or alerted remotely, such as by one or more of the input/output devices 175.

The apparatus 180 may accordingly be worn by a subject, such as on a wrist, arm, or other body part, and may be remote from analytical hardware and software, as generally illustrated in FIG. 1. The apparatus 180 may be arranged to communicate with the processor 165 over a wired or wireless communication mechanism, and may have continuous or asynchronous communication with the processor 165.

The apparatus 180 may be a low power system. Power for the pump 140, sensor 125, alarm 160, or combinations of those components, may be obtained completely or partially from a power source 150 that may be included in the apparatus 150. The pumping, sensing, and analysis operations described, may run continuously, or for discrete periods of time, for example every five minutes, to monitor dehydration status. The power source 150 may be implemented as a battery, solar panel, or a passive power source harvested from the subject itself. For example, the power source 150 may generate power based on physical activity of the subject. One example of such a power source is a regenerative braking knee brace that may deliver up to 5 Watts of power, which is described at J. M. Donelan et al, “Biomechanical Energy Harvesting: Generating Electricity During Walking with Minimal User Effort,” Science 319(5864), pp. 807-810 (2008), and incorporated by reference herein in its entirety for any purpose.

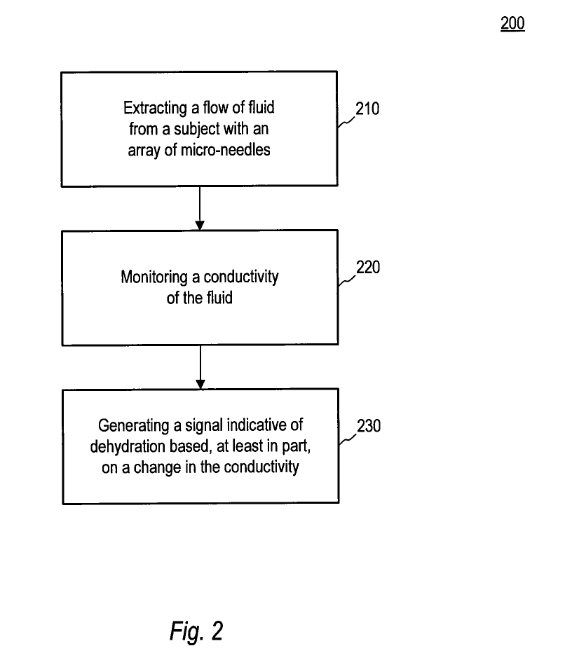

FIG. 2 is a flowchart of an example process (200) of monitoring a subject for dehydration in accordance with at least some embodiments of the present disclosure. Process 200 may include one or more functions, operations or actions as illustrated by blocks 210, 220 and/or 230. Processing may begin at block 210. In block 210, a flow of fluid may be extracted from a subject with an array of micro-needles. Block 210 may be followed by block 220. In block 220, the conductivity of the fluid may be monitored. Block 220 may be followed by block 230. In block 230, a signal indicative of dehydration may be generated based, at least in part, on a change in the conductivity of the fluid.

As generally described above (See discussion for FIG. 1), the extraction of fluid in block 210 may occur by a pumping force, or by pressure-driven flow that may in part be aided by the fluid pressure of the subject. The flow may be continuous, and in some embodiments the continuous flow may occur only for a period of time, such as continuous flow initiated every five minutes, and stopped between measurement intervals. The fluid may include blood, plasma, sweat, interstitial fluid, or combinations of those fluids.

The conductivity of the fluid may be monitored in block 220 by a conductivity sensor in fluid communication with the array of micro-needles. The conductivity of the fluid may be monitored continuously, or intermittently.

The signal indicative of dehydration may be generated in block 230 when a change in the measured conductivity is determined to be greater than a noise level, a threshold level, or both. The signal indicative of dehydration may be generated when the conductivity is determined to have decreased more than a noise level, a threshold level, or both. In general, the conductivity of sweat may vary from person to person in a range from about 2 mS/cm to 12 mS/cm. For an early warning of dehydration, some examples provide a signal indicative of dehydration when there is a body mass loss of about 1 to about 2 percent or less. Dehydration may not be realized by a subject until a greater amount of body mass loss. In some examples, the signal indicative of dehydration may be provided at a higher percentage of body mass loss. A body mass loss of about 1 percent may correlate to an increase in salt content of sweat of about 10 percent. Accordingly, in one example where the conductivity of sweat is measured, a signal indicative of dehydration may be generated when the conductivity has changed by about 10 percent or more in some examples, by about 5 percent or more in some examples, or by about 2 percent or more in some examples, or by about 1 percent or more in some examples.

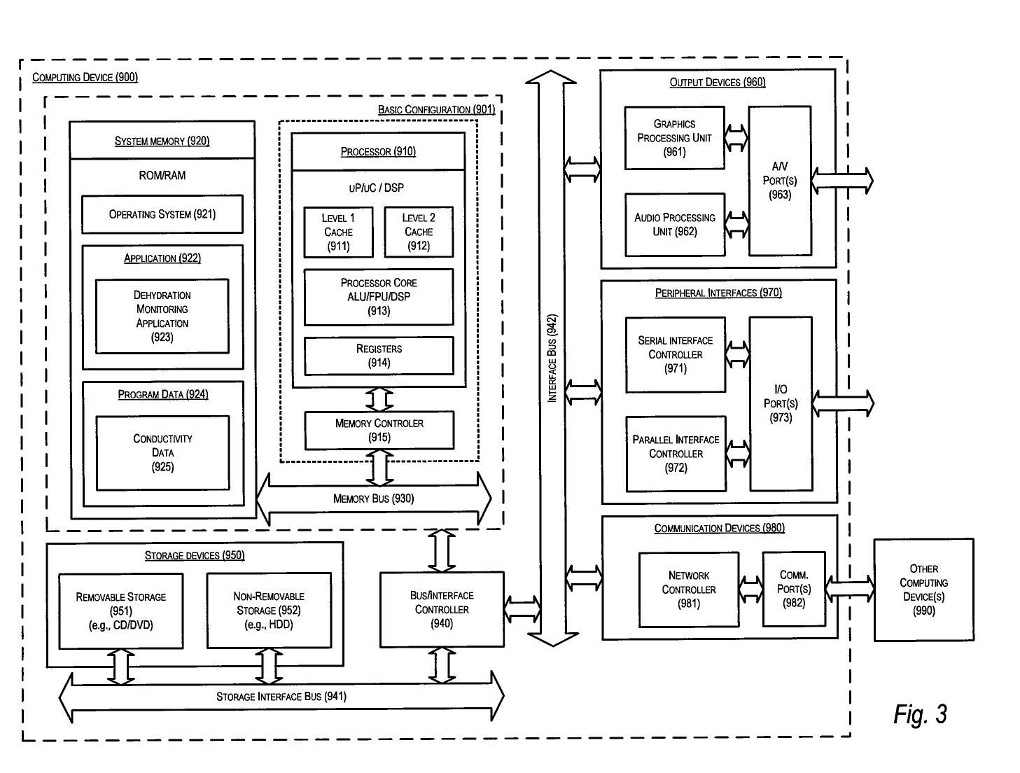

FIG. 3 is a block diagram illustrating an example computing device 900 that is arranged for monitoring for dehydration in accordance with at least some embodiments of the present disclosure. The computing device 900 may be substituted for all or a portion of the processor 165, memory 170, and input/output devices 175 of FIG. 1. In a very basic configuration 901, computing device 900 typically includes one or more processors 910 and system memory 920. A memory bus 930 may be used for communicating between the processor 910 and the system memory 920.

Depending on the desired configuration, processor 910 may be of any type including but not limited to a microprocessor (μP), a microcontroller (μC), a digital signal processor (DSP), or any combination thereof. Processor 910 may include one more levels of caching, such as a level one cache 911 and a level two cache 912, a processor core 913, and registers 914. An example processor core 913 may include an arithmetic logic unit (ALU), a floating point unit (FPU), a digital signal processing core (DSP Core), or any combination thereof. An example memory controller 915 may also be used with the processor 910, or in some implementations the memory controller 915 may be an internal part of the processor 910.

Depending on the desired configuration, the system memory 920 may be of any type including but not limited to volatile memory (such as RAM), non-volatile memory (such as ROM, flash memory, etc.) or any combination thereof. System memory 920 may include an operating system 921, one or more applications 922, and program data 924. Application 922 may include a dehydration monitoring application 923 that is arranged to receive signals indicative of a conductivity of a fluid and identify a change in the signals indicative of dehydration, and may generate a signal indicative of dehydration. Program Data 924 may include conductivity data 925 used by the dehydration monitoring application 923. In some embodiments, application 922 may be arranged to operate with program data 924 on an operating system 921 in accordance with one or more of the techniques, methods and/or processes described herein. This described basic configuration is illustrated in FIG. 3 by those components within dashed line 901.

Computing device 900 may have additional features or functionality, and additional interfaces to facilitate communications between the basic configuration 901 and any required devices and interfaces. For example, a bus/interface controller 940 may be used to facilitate communications between the basic configuration 901 and one or more data storage devices 950 via a storage interface bus 941. The data storage devices 950 may be removable storage devices 951, non-removable storage devices 952, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 920, removable storage 951 and non-removable storage 952 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 900. Any such computer storage media may be part of device 900.

Computing device 900 may also include an interface bus 942 for facilitating communication from various interface devices (e.g., output interfaces, peripheral interfaces, and communication interfaces) to the basic configuration 901 via the bus/interface controller 940. Example output devices 960 include a graphics processing unit 961 and an audio processing unit 962, which may be configured to communicate to various external devices such as a display or speakers via one or more A/V ports 963.

Example peripheral interfaces 970 include a serial interface controller 971 or a parallel interface controller 972, which may be configured to communicate with external devices such as input devices (e.g., keyboard, mouse, pen, voice input device, touch input device, etc.) or other peripheral devices (e.g., printer, scanner, etc.) via one or more I/O ports 973.

An example communication device 980 includes a network controller 981, which may be arranged to facilitate communications with one or more other computing devices 990, or an apparatus such as the apparatus 180 of FIG. 1, over a network communication link via one or more communication ports 982.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 900 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 900 may also be implemented as a personal computer including both laptop computer and non-laptop computer configurations.

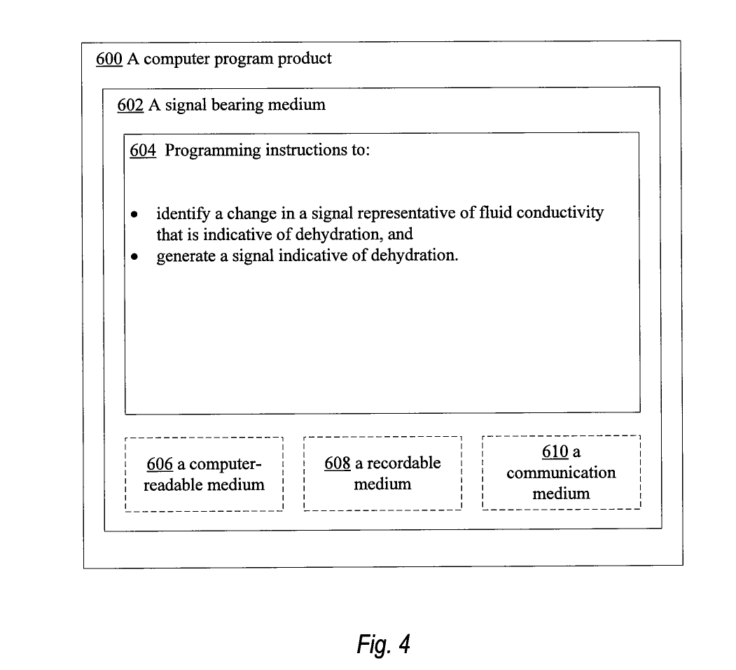

FIG. 4 is a block diagram illustrating an example computer program product 600 that is arranged to store instructions for monitoring a fluid for dehydration in accordance with at least some embodiments of the present disclosure. The computer readable medium 602, which may be implemented as or include a computer-readable medium 606, a recordable medium 608, a communications medium 610, or combinations thereof, stores instructions 604 that may configure a processing unit to perform all or some of the processes previously described. The computer readable medium 602 may be implemented as a memory. These instructions may include, for example, one or more executable instructions for identifying a change in a signal representative of fluid conductivity that is indicative of dehydration. The instructions may include comparing a magnitude of a detected change or a noise level, a threshold level, or both. The instructions may further include generating a signal indicative of a dehydration condition.

The present disclosure is not to be limited in terms of the particular examples described in this application, which are intended as illustrations of various aspects. Many modifications and examples can may be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and examples are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular examples only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 items refers to groups having 1, 2, or 3 items. Similarly, a group having 1-5 items refers to groups having 1, 2, 3, 4, or 5 items, and so forth.

While the foregoing detailed description has set forth various examples of the devices and/or processes via the use of block diagrams, flowcharts, and/or examples, such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one example, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the examples disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. For example, if a user determines that speed and accuracy are paramount, the user may opt for a mainly hardware and/or firmware vehicle; if flexibility is paramount, the user may opt for a mainly software implementation; or, yet again alternatively, the user may opt for some combination of hardware, software, and/or firmware.

In addition, those skilled in the art will appreciate that the mechanisms of the subject matter described herein are capable of being distributed as a program product in a variety of forms, and that an illustrative example of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

While various aspects and examples have been disclosed herein, other aspects and examples will be apparent to those skilled in the art. The various aspects and examples disclosed herein are for purposes of illustration and are not intended to be limiting, with the true scope and spirit being indicated by the following claims.

What is claimed is:

1. An apparatus for use in detecting dehydration, the apparatus comprising:
an array of micro-needles;
a capillary coupled to the array of micro-needles and configured to receive fluid from at least a portion of the array of micro-needles;
a conductivity sensor arranged to measure conductivity of the fluid within the capillary, wherein a change in conductivity of the fluid is indicative of dehydration; and
a pump positioned to move fluid through the capillary.

2. The apparatus of claim 1 wherein the fluid comprises plasma.

3. The apparatus of claim 1 further comprising a plurality of capillaries, each configured to receive fluid from the array of micro-needles.

4. The apparatus of claim 1 wherein the conductivity sensor comprises a pair of electrodes.

5. The apparatus of claim 4 wherein the pair of electrodes are embedded within the capillary.

6. The apparatus of claim 1 wherein the pump comprises an electroosmotic pump.

7. The apparatus of claim 6 wherein the electroosmotic pump comprises a pair of electrodes.

8. The apparatus of claim 1 further comprising a passive power source coupled to the conductivity sensor.

9. The apparatus of claim 1 wherein an inlet of the capillary contains fabric configured to filter fluid entering the capillary.

10. The apparatus of claim 1 wherein the pump is configured for continuous flow.

11. A system for detecting dehydration in a live subject, the system comprising:
a micro-needle assembly comprising:
an array of micro-needles configured to draw fluid from the live subject;
a conductivity sensor positioned to measure a conductivity of the fluid; and
a pump configured to draw the fluid toward the conductivity sensor; and
a processor configured to receive conductivity measurements from the conductivity sensor, identify a change in the conductivity based at least in part on the conductivity measurements, and provide an indication of dehydration based at least in part on the identified change in the conductivity.

12. The system of claim 11 wherein the micro-needle assembly is worn by the subject.

13. The system of claim 11 further comprising an alarm arranged to receive the indication from the processor and provide a signal indicative of dehydration to the live subject.

14. The system of claim 11 further comprising a memory coupled to the conductivity sensor and configured to store the conductivity measurements.

15. The system of claim 14 wherein the processor is configured to receive the conductivity measurements from the memory.

16. A method for monitoring a subject for dehydration, the method comprising:
extracting a flow of fluid from the subject with an array of micro-needles;
monitoring the conductivity of the fluid; and
generating a signal indicative of dehydration responsive at least in part to a change of the conductivity of the fluid.

17. The method according to claim 16 wherein the fluid comprises plasma.

18. The method according to claim 16 wherein monitoring the conductivity of the fluid comprises applying a voltage across at least a portion of the fluid.

19. The method according to claim 16 further comprising generating an alarm signal based at least in part on the signal indicative of dehydration.

20. The method according to claim 16 wherein monitoring the conductivity of the fluid comprises continuously pumping the fluid across a conductivity sensor.
